(19) United States
(12) Patent Application Publication
(10) Pub. No.: US 2024/0337651 A1
(43) Pub. Date: Oct. 10, 2024

(54) METHODS AND KITS FOR DETECTING CANNABINOIDS

(71) Applicants: Ricovr Healthcare Inc., Princeton, NJ (US); Indian Institute of Technology Madras (IIT Madras), Chennai (IN)

(72) Inventors: Venkata Raghavendra Sai Vemulakonda, Chennai (IN); Divagar Murugan, Chennai (IN); Himanshu Bhatia, Skillman, NJ (US); Joseph Walter Seimetz, Princeton, NJ (US); Elana Michelle Edery, Highland Park, NJ (US)

(73) Assignees: Ricovr Healthcare Inc., Princeton, NJ (US); Indian Institute of Technology Madras (IIT Madras), Chennai (IN)

(21) Appl. No.: 18/631,994

(22) Filed: Apr. 10, 2024

Related U.S. Application Data
(60) Provisional application No. 63/495,181, filed on Apr. 10, 2023.

(51) Int. Cl. G01N 33/543; G01N 21/75; G01N 33/68

(52) U.S. Cl. CPC G01N 33/54346; G01N 21/75; G01N 33/68; G01N 2021/755; G01N 2021/757; G01N 2333/765; G01N 2333/77

(57) ABSTRACT

The disclosure provides methods of detecting a cannabinoid or metabolite thereof in a sample and test kits for the same. For example, the cannabinoid such as tetrahydrocannabinol (THC) bound to bovine serum albumin (BSA) may induce changes in optical properties of an optical biosensor employed for the said detection wherein the optical biosensor is immobilized with a suitable bioreceptor or through interactions with a bioreceptor-nanoparticle conjugate.

### CROSS-REFERENCE TO RELATED APPLICATION

This application claims priority to and the benefit of U.S. Provisional Application No. 63/495,181, filed Apr. 10, 2023, the content of which is incorporated herein by reference in its entirety.

### FIELD OF INVENTION

The disclosure relates to a method and a test kit for detecting a cannabinoid or a metabolite thereof in a sample.

### BACKGROUND

Cannabis, which is the most-used illegal drug, is commonly sourced from Cannabaceae, a flowering plant that includes various species, notably Cannabis sativa and Cannabis indica. A wide variety of cannabinoids such as psychoactive Δ9-Tetrahydrocannabinol (THC), and non-psychoactive cannabidiol (CBD) and cannabinol (CBN) influence or impairs psychomotor and cognitive functions in a human or animal body. Hence, the use of cannabis is associated with a considerable number of traffic accidents worldwide.

Due to legalization in countries like Canada and in some states in the United States, a need for quick and accurate THC tests may rise, for example, for testing driving under influence (DUI) of cannabis drug or regulating workplace drug consumption.

The effects of cannabis vary among individuals and gets absorbed by lipids and fats. Subsequently, it is metabolized in the liver into psychoactive 11-hydroxy Δ9-THC (11-OH-THC) and later oxidized to non-psychoactive 11-nor-9carboxy Δ9-THC (THC-COOH) (Rosado et al. 2017). Several studies have reported the use of traditional methods such as high-performance liquid chromatography, capillary electrophoresis, microplate ELISA, radial immunoassay, gas chromatography, mass spectroscopy, and its combination in detecting the psychoactive Δ9-THC as well as the secondary metabolites in various samples, including blood, plasma, serum, oral saliva, urine, exhaled breath, and hair. Though the traditional methods are sensitive in detecting the constituents, its sophistication, requirement of personnel with significant expertise in operation and laboratory setup, elaborate sample processing protocols, and longer processing time had limited its widespread utility.

Based on previous studies, detecting Δ9-THC in oral saliva has become a potential test to identify the subjects under the influence of cannabis. However, the test still possesses specific challenges. One big challenge in realizing a THC test could be the false-positive results from subjects passively exposed to cannabis smoke. However, it purely depends on the duration of the exposure, the amount of THC in the smoke, and the enclosed smoking area (Hayley et al. 2018). Studies were reported testing subjects passively exposed to cannabis smoke and revealed a range of about 0-1.2 ng/ml of THC in the saliva samples after 1.5 hrs of passive exposure (Niedbala et al. 2005). Another study reports 70% of the subjects exposed passively to THC for 3 hrs, showed an oral THC concentration of 2 ng/ml of THC, which the current confirmatory cut-off concentration, despite, there is no trace of THC-COOH (Moore et al. 2011). A further critical concern is the subjects who have consumed hemp products with a trace amount of Δ9-THC. Hayley et al., in 2018, conducted a study with subjects who consumed THC infused oil at two different concentrations include 10 mg/kg (Low-THC) and 20 mg/kg (High-THC content). The results were analyzed using Securetec Drugwipe® II twin device at a cut-off level of 20 ng/ml, and the results revealed that no THC was found in the oral fluid even after 240 mins of ingestion (Hayley et al. 2018).

Even with all these challenges test for Δ9-THC in oral saliva remain as a potential method and various diagnostic devices were developed for the particular application. The Draeger Drug test 5000® is one of the commercial devices that detects salivary THC and has been approved by the Canadian government for roadside testing of salivary THC. This, however, is limited by lack of sensitivity for THC concentrations less than 5 ng/ml. It is further characterized by high false positives and false negative rate up to 13.4% and 14.5%, respectively (He et al. 2020). Other test devices such as Securetec Drugwipe® 5+, DDS®, and RapidSTAT® are limited to accomplish recommended sensitivity of >80%, selectivity, and accuracy (Strano-Rossi et al. 2012; Arkell et al. 2019). Hence, these have not been found suitable for on-site detection of Δ9-THC. Also, a recent review of nine different devices including Oraline®, Cozart DDS 806®, BIOSENSE Dynamic®, OraLAb®, OrAlert®, Oratect III®, Drugwipe®, DrugTest 5000®, and RapidSTAT® concluded that all these devices fail to satisfy the minimum requirements prescribed by ROSITA, ROSITA II and DRUID research projects (Dobri et al. 2019). Other previously reported assays such as a lateral flow assay based technique to detect THC obtained an LoD of about 2 ng/ml in artificial saliva samples (Thapa et al. 2020). However, the setup demands a bulkier read-out unit.

In view of the above, the demand for a device similar to a breath analyzer for estimating blood alcohol content is of high priority. The present disclosure addresses the said need.

### SUMMARY

Provided herein, inter alia, methods of detecting a cannabinoid or metabolite thereof in a sample and test kits for the same.

Particularly, the present disclosure provides a method of detecting presence or absence of a cannabinoid or metabolite thereof in a biological sample, comprising:
contacting the biological sample with nanoparticle conjugated bioreceptor(s) capable of binding the cannabinoid or metabolite thereof, wherein
   a) the nanoparticle conjugated bioreceptor(s) are either immobilized on a fiber optic sensor, or
   b) the nanoparticle conjugated bioreceptor(s) are in solution phase; and
detecting a change in optical property of the fiber optic sensor in response to
a) direct binding of the cannabinoid or metabolite thereof to the nanoparticle conjugated bioreceptor(s) immobilized on the fiber optic sensor surface, or
b) binding of free nanoparticle conjugated bioreceptor(s) in solution phase to cannabinoid or metabolite thereof immobilized on a fiber optic sensor surface.

In some embodiments, the biological sample is selected from a group comprising blood, plasma, serum, saliva, tears and urine from a subject.

In some embodiments, the biological sample is optionally diluted.

In some embodiments, the dilution solution comprises about 10 to 100 mM Tris, about 10 to 200 mM boric acid, and about 1 to 20 mM EDTA in water and optionally about 10 to 300 mM NaCl and/or about 0.1 to 0.5 wt % lipid polymer stabilizer in water.

In some embodiments, the cannabinoid or metabolite thereof is selected from a group comprising tetrahydrocannabinol (THC), cannabidiol (CBD) and cannabinol (CBN) or any combination thereof.

In some embodiments, the bioreceptor is selected from a group comprising a peptide, a protein, antibody, an aptamer, a polymer, a hapten, a small molecule, a nucleic acid and an oligosaccharide or any combination thereof.

In some embodiments, the cannabinoid or metabolite thereof immobilized on the fiber optic sensor surface is immobilized in the form of a cannabinoid/metabolite-carrier conjugate. In some embodiments, the carrier is selected from a group comprising Bovine Serum Albumin (BSA), serum globulins, albumins and ovalbumin or any combination thereof.

In some embodiment, the method of detecting presence or absence of a cannabinoid or metabolite thereof in a biological sample comprises:
contacting the biological sample with nanoparticle conjugated bioreceptor(s) capable of binding the cannabinoid or metabolite thereof, wherein the nanoparticle conjugated bioreceptor(s) are in solution phase; and
detecting a change in optical property of the fiber optic sensor in response to binding of free nanoparticle conjugated bioreceptor(s) in solution phase to cannabinoid or metabolite thereof immobilized on a fiber optic sensor surface.

In some embodiments, the change in optical property of the fiber optic sensor is measured using a device comprising:
i) a light source located proximal to one end of the optical fiber; and
ii) a detector located proximal to another end of the optical fiber, wherein the detector is configured to sense the change in the optical property of light that traverses through the optical fiber when the probe region is exposed to the biological sample comprising the cannabinoid or metabolite thereof.

The present disclosure further provides a test kit for detecting a cannabinoid or metabolite thereof in a biological sample, comprising:
the device is as described above;
means for sample collection and optionally, quantification and/or storage;
and one or more of:
a vial or container comprising a blocking agent;
a second vial or container comprising a sample diluting solution or components thereof;
and
an instruction manual.

Also envisaged herein is use of the biosensor or the test kit described above for the qualitative and/or quantitative detection of cannabinoid or metabolite thereof in a biological sample.

Other aspects of the invention are disclosed infra.

### BRIEF DESCRIPTION OF THE DRAWINGS

FIG. 1A to 1B show temporal response (FIG. 1A) and absorbance response (FIG. 1B) obtained by performing competitive fiber optic immunoassay using HIgG as a model analyte.

FIG. 2A-2B show temporal response (FIG. 2A) and absorbance response (FIG. 2B) obtained by performing competitive fiber optic immunoassay using THC as analyte (no. of experiments, n=3). The control probe has greater standard deviation in comparison to the other concentrations tested. However, with the THC concentration of 1000 pg/ml or greater, the sensor does not show any response as a drop in intensity.

FIG. 3 shows simulated results obtained depending on number of AuNP labels that could be blocked with a given THC concentration.

FIG. 4A-4B show temporal response obtained from competitive fiber optic immunoassay using varying size of AuNP at 10× concentration with respect to varying THC concentration (n=2).

FIG. 5A shows temporal response and FIG. 5B shows absorbance response obtained from competitive fiber optic immunoassay using 20 nm AuNP label at 1× concentration with respect to varying THC concentration (n=3). NSA corresponds to non-specific adsorption. It is difficult to distinguish 5 and 10 ng/ml, however 5 and 50 ng/ml are distinguishable.

FIG. 6 shows temporal response obtained from competitive fiber optic immunoassay using 30 nm AuNP label at 1× concentration with respect to varying THC concentration. (n=2).

FIG. 7 shows an exemplary embodiment of label free immunoassay for the detection of THC.

FIG. 8 shows temporal response obtained from anti-THC antibody immobilized LSPR probe (OD ˜2) due to binding of THC molecules.

FIG. 9 shows that THC measurements in users according to time (hours) after THC ingestion.

FIG. 10 shows changes of absorbance values when human saliva samples were spiked with different amounts of THC.

FIG. 11 shows changes temporal response obtained from U-bent fiber optic sensor probe (left) due to binding of AuNP conjugates of varying size and concentration and (right) due to binding of 20 nm AuNPs at 10× concentration with (1 ng/ml) and without analyte, THC (0 ng/ml).

### DETAILED DESCRIPTION

### Definitions

As used herein, the term “biological sample” or “sample” encompasses a variety of sample types, including blood and other liquid samples of biological origin (e.g., blood, plasma, serum, gastrointestinal secretions, homogenates of tissues or tumors, synovial fluid, feces, saliva, sputum, cyst fluid, amniotic fluid, cerebrospinal fluid, peritoneal fluid, lung lavage fluid, semen, lymphatic fluid, tears, and prostatic fluid), solid tissue samples such as a biopsy specimen or tissue cultures, or cells derived therefrom and the progeny thereof. The term also encompasses various kinds of clinical samples obtained from any species, and also includes cells in culture, cell supernatants, and cell lysates. In particular, target molecules present in a biological sample are indicative consumption or intake of cannabinoid, derivative thereof, or metabolite thereof processed in a subject.

The term “cannabinoid” as used herein refers to a class of compounds obtained (e.g., isolated) from cannabis plants. Exemplary cannabinoids may include, but not be limited to, tetrahydrocannabinol (THC), cannabidiol (CBD) and cannabinol (CBN) or derivatives thereof.

The term “metabolite” as used herein refers to a substance or molecular derivative resulted from biological decomposition, modification or alteration of a compound, which may occur after biological consumption or intake of the compound in a cell, tissue, organs or body. In some embodiments, the “cannabinoid metabolites” or equivalent terms refer to substances resulted, isolated or obtained from a human or a subject intake or consumption of the cannabinoid compounds. The cannabinoid compounds may be synthetic or naturally obtained products and may be administered for medical use or leisure purpose.

Cannabinoids and metabolites, in the context of the present disclosure, have been interchangeably referred to as ‘analyte’ or ‘analyte of interest’ or ‘target biomolecule’.

The term “bioreceptor” as used herein refers to a substance, e.g., a peptide, a protein, antibody, an aptamer, a polymer, hapten, a small molecule, a nucleic acid, an oligosaccharide, a cell, or the like, that specifically interact, associate, recognize and/or bind to a biological molecule of interest or analyte (e.g., target molecule). The bioreceptor may also involve non-specific interaction with other molecules such as the probe of an optical fiber, other components in the biological samples, or with blocking (calibrating) agent.

The term “nanoparticle conjugated bioreceptor(s)” or equivalent terms thereof refers to bioreceptors bound to nanoparticle, wherein a single nanoparticle (NP) may comprise multiple bioreceptors bound to its surface. The term ‘label’ or ‘NP-label’ has been used interchangeably for nanoparticle conjugated bioreceptor(s) in solution phase.

The term “probe”, in the context of the present disclosure, refers to a portion of the optical fiber that enables to study, investigate, or detect the analyte of interest in a sample. In some embodiments, the probe may be designed interact or associate with the analyte or a bioreceptor bound to the analyte, leading to changes in one or more of its optical properties such that the presence of the analyte (e.g., target molecule) in the sample may be studied or recognized. In the context of the present disclosure, the probe is a fiber optic probe.

The term “associating with” or “associated with” in the context of a bioreceptor or bioreceptor function associating with (or associated with) other substance (e.g., probe) means that the bioreceptor is involved in an interaction with the other substance (e.g., probe) directly or indirectly, or entirely or partially. Also, as used herein, what is described as associating or being associated with the other substance means chemically and/or physically interact with the other substance (e.g., probe).

The term “optical property” as used herein refers to a property of material in response to irradiation of light or interaction with light (e.g., UV, infra-red, or visible light having wave length of about 400 to 800 nm). Exemplary optical properties include refractive index, dispersion, transmittance and transmission coefficient, absorption, scattering, turbidity, reflectance and reflectivity (reflection coefficient), albedo, perceived color, fluorescence, phosphorescence, photoluminescence, optical bistability, dichroism, birefringence, optical activity, and photosensitivity. In some embodiments, the optical intensity changes (e.g., absorption, photoluminescence, or fluorescence) at the photodetector may be in real-time at a specific wavelength.

The term “nanoparticle” as used herein refers to a particular substance having a maximum diameter in a range of about 1 to 999 nm, about 1 to 900 nm, about 1 to 800 nm, about 1 to 700 nm, about 1 to 600 nm, about 1 to 500 nm, about 1 to 400 nm, about 1 to 300 nm, about 1 to 200 nm, about 1 to 100 nm, or particularly of about 1 to 50 nm. In certain embodiments, the nanoparticle may have a maximum dimeter of about 10 nm, about 20 nm, about 30 nm, about 40 nm, or about 50 nm. In certain embodiments, the nanoparticle includes core substance made of gold, silver, silica, or combinations thereof and shell composition such a polymer, small molecule, coating material or other biological material (e.g., antibody, peptide, nucleic acid, antigen, protein, lipid or the like). In certain embodiments, the nanoparticle may have a suitable shape, e.g., spheres, rods, cubes, stars, polygons, and the like, but not limited thereto. The shape of the nanoparticle may be determined during the process of manufacturing and may be controlled based on physical or chemical properties of the material thereof or other additive on its surface.

The term “blocking agent” as used herein refers to a component (e.g., protein, lipids, polymers or small molecules) in a mixture system (e.g., mixture of bioreceptor, target molecule and/or probes) to prevent non-specific binding among other species while improving the specific binding affinity between specifically binding molecules. The blocking agent may be added in saturating concentration, does not interact with the specifically binding molecules, and maintains the background level from surrounding molecules or environment minimum. In this context, the term “calibration agent” as used herein may be interchangeably used as the calibrating agent in the test kit or in the device stabilizes or minimizes background signal (e.g., optical signal or change in optical properties in the probe).

The term “antibody” (Ab) as used herein includes monoclonal antibodies, polyclonal antibodies, multi-specific antibodies (for example, bispecific antibodies and polyreactive antibodies), and antibody fragments. Thus, the term “antibody” as used in any context within this specification is meant to include, but not be limited to, any specific binding member, immunoglobulin class and/or isotype (e.g., IgG1, IgG2, IgG3, IgG4, IgM, IgA, IgD, IgE); and biologically relevant fragment or specific binding member thereof, including but not limited to Fab, F(ab′)2, Fv, and scFv (single chain or related entity). It is understood in the art that an antibody is a glycoprotein comprising at least two heavy (H) chains and two light (L) chains inter-connected by disulfide bonds, or an antigen binding portion thereof. A heavy chain is comprised of a heavy chain variable region (VH) and a heavy chain constant region (CH1, CH2 and CH3). A light chain is comprised of a light chain variable region (VL) and a light chain constant region (CL). The variable regions of both the heavy and light chains comprise framework regions (FWR) and complementarity determining regions (CDR). The four FWR regions are relatively conserved while CDR regions (CDR1, CDR2 and CDR3) represent hypervariable regions and are arranged from NH2 terminus to the COOH terminus as follows: FWR1, CDR1, FWR2, CDR2, FWR3, CDR3, FWR4. The variable regions of the heavy and light chains contain a binding domain that interacts with an antigen while, depending of the isotype, the constant region(s) may mediate the binding of the immunoglobulin to host tissues or factors. The term “monoclonal antibody” as used herein refers to an antibody obtained from a population of substantially homogeneous antibodies, i.e., the individual antibodies comprising the population are identical except for possible naturally occurring mutations that may be present in minor amounts. The term “polyclonal antibody” refers to preparations that include different antibodies directed against different determinants (“epitopes”). Also included in the definition of “antibody” as used herein are chimeric antibodies, humanized antibodies, and recombinant antibodies, human antibodies generated from a transgenic non-human animal, as well as antibodies selected from libraries using enrichment technologies available to the artisan. In certain embodiments, the antibody having specificity toward cannabinoids or metabolites (e.g., THC) thereof may be used as bioreceptors.

As used herein, the term ‘comprising’ when placed before the recitation of steps in a method means that the method encompasses one or more steps that are additional to those expressly recited, and that the additional one or more steps may be performed before, between, and/or after the recited steps. For example, a method comprising steps a, b, and c encompasses a method of steps a, b, x, and c, a method of steps a, b, c, and x, as well as a method of steps x, a, b, and c. Furthermore, the term “comprising” when placed before the recitation of steps in a method does not (although it may) require sequential performance of the listed steps, unless the content clearly dictates otherwise. For example, a method comprising steps a, b, and c encompasses, for example, a method of performing steps in the order of steps a, c, and b, the order of steps c, b, and a, and the order of steps c, a, and b, etc.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The suffix ‘(s)’ at the end of any term in the present disclosure envisages in scope both the singular and plural forms of said term.

As used in this specification and the appended claims, the singular forms ‘a’, ‘an’ and ‘the’ includes both singular and plural references unless the content clearly dictates otherwise.

Unless defined otherwise, all technical and scientific terms used herein have the same meanings as commonly understood by one of ordinary skill in the art.

Numerical ranges stated in the form ‘from x to y’ include the values mentioned and those values and sub-ranges that lie within the range of the respective measurement as known to the skilled person. If several preferred numerical ranges are stated in this form, of course, all the ranges formed by a combination of the different end points are also included.

The use of the expression ‘at least’ or ‘at least one’ suggests the use of one or more elements or ingredients or quantities, as the use may be in the embodiment of the disclosure to achieve one or more of the desired objects or results. As such, the terms “a” (or “an”), “one or more”, and “at least one” can be used interchangeably herein.

The terms ‘about’ or ‘approximately’ as used herein when referring to a measurable value such as a parameter, an amount, a temporal duration, and the like, are meant to encompass variations of and from the specified value, such as variations of +/−10% or less, +1-5% or less, +/−1% or less, and +/−0.1% or less of and from the specified value, insofar such variations are appropriate to perform in the disclosed invention. It is to be understood that the value to which the modifier ‘about’ or ‘approximately’ refers is itself also specifically, and preferably, disclosed.

As used herein, the terms ‘include’, ‘have’, ‘comprise’, ‘contain’ etc. or any form of said terms such as ‘having’, ‘including’, ‘containing’, ‘comprising’ or ‘comprises’ are inclusive and will be understood to imply the inclusion of a stated element, integer or step, or group of elements, integers or steps, but not the exclusion of any other element, integer or step, or group of elements, integers or steps.

The use of any and all examples, or exemplary language (e.g., “such as”) provided with respect to certain embodiments herein is intended merely to better illustrate the disclosure and does not pose a limitation on the scope of the disclosure otherwise claimed.

As regards the embodiments characterized in this specification, it is intended that each embodiment be read independently as well as in combination with another embodiment. For example, in case of an embodiment 1 reciting 3 alternatives A, B and C, an embodiment 2 reciting 3 alternatives D, E and F and an embodiment 3 reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Reference throughout this specification to “some embodiments”, “one embodiment”, “an embodiment”, “a preferred embodiment”, “a non-limiting embodiment” or “an exemplary embodiment” means that a particular feature, structure or characteristic described in connection with the embodiment may be included in at least one embodiment of the present disclosure. Thus, the appearances of the phrases “in some embodiments”, “in one embodiment”, “in an embodiment”, “a preferred embodiment”, “a non-limiting embodiment” or “an exemplary embodiment” in various places throughout this specification may not necessarily all refer to the same embodiment. It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination.

Throughout this specification, the term ‘a combination thereof’, ‘combinations thereof’ or ‘any combination thereof’ or ‘any combinations thereof’ are used interchangeably and are intended to have the same meaning, as regularly known in the field of patent disclosures.

### Method(s) of Detection

In line with the objective of devising a method for accurate detection of cannabinoid or metabolite thereof in a sample, the present disclosure provides method(s) of detecting presence or absence of a cannabinoid or metabolite thereof in a biological sample derived from a subject based on use of an optical biosensor.

Particularly, the present disclosure provides a method of detecting presence or absence of a cannabinoid or metabolite thereof in a biological sample, comprising:
contacting the biological sample with nanoparticle conjugated bioreceptor(s) capable of binding the cannabinoid or metabolite thereof, wherein
   a) the nanoparticle conjugated bioreceptor(s) are either immobilized on a fiber optic sensor, or
   b) the nanoparticle conjugated bioreceptor(s) are in solution phase; and
detecting a change in optical property of the fiber optic sensor in response to
a) direct binding of the cannabinoid or metabolite thereof to the nanoparticle conjugated bioreceptor(s) immobilized on the fiber optic sensor surface, or
b) binding of free nanoparticle conjugated bioreceptor(s) in solution phase to cannabinoid or metabolite thereof immobilized on a fiber optic sensor surface.

In some embodiments, the cannabinoid or metabolite thereof includes, but is not particularly limited to, tetrahydrocannabinol (THC), cannabidiol (CBD) and cannabinol (CBN) or any combination thereof. Derivatives of the said compounds are also envisaged in the scope of the present disclosure.

In a preferred embodiment, the cannabinoid or metabolite thereof is THC or any derivative thereof.

In some embodiments, the biological sample is selected from a group comprising blood, plasma, serum, saliva, urine, sweat and tears from a subject. The biological sample may be obtained directly or indirectly from the subject (e.g., human). Saliva as a sample is of particular importance as it is easy to collect in public, non-invasive, suitable for a roadside test and the concentration of THC correlates with cognitive impairment.

Thus, in a preferred embodiment, the biological sample comprises saliva obtained from a subject.

In some embodiments, the biological sample is optionally diluted.

In some embodiments, the biological sample comprises saliva obtained from a subject; and the biological sample is optionally diluted.

In some embodiments, the saliva is diluted in a dilution medium.

In some embodiments, the biological sample is optionally diluted before being subjected to the above described method.

In some embodiments, the biological sample may be diluted using a diluting solution, wherein the diluting solution may include one or more of salts, weak acids, weak bases, reducing agents, proteases, protease inhibitors and detergents.

In a non-limiting embodiment, some examples of salts, weak acids and weak bases employable in the diluting solution include Sodium chloride, potassium chloride, sodium phosphate, potassium phosphate, hydrogen chloride, sodium hydroxide, tris(hydroxymethyl)aminomethane and borate or any combination thereof.

In another non-limiting embodiment, some examples of reducing agents employable in the diluting solution include dithiothreitol and mercaptoethanol or a combination thereof.

In a further non-limiting embodiment, an example of employable chelating agent employable in the diluting solution includes ethylenediaminetetraacetic acid and other known alternatives thereof.

In yet another non-limiting embodiment, some examples of detergents employable in the diluting solution include tween, triton and tergitol or any combination thereof.

In some embodiments, the saliva dilution solution is based on a Tris-borate-EDTA (TBE) buffer.

In some embodiments, the dilution solution comprises Tris, boric acid, EDTA and optionally, salt and/or a lipid polymer stabilizer.

In some embodiments, the dilution solution comprises Tris, boric acid, EDTA and NaCl.

In some embodiments, the dilution solution comprises Tris, boric acid, EDTA and a lipid polymer stabilizer.

In some embodiments, the dilution solution comprises Tris, boric acid, EDTA, NaCl and a lipid polymer stabilizer.

In some embodiments, the dilution solution comprises about 10 to 100 mM Tris, about 10 to 200 mM boric acid, and about 1 to 20 mM EDTA in water or aqueous solution.

In a preferred embodiment, the dilution solution includes about 100 mM Tris, about 90 mM boric acid, and about 10 mM EDTA in water or aqueous solution.

In some embodiments, the dilution solution comprises about 10 to 100 mM Tris, about 10 to 200 mM boric acid, about 1 to 20 mM EDTA and optionally about 10 to 300 mM salt and/or about 0.1 to 0.5 wt % lipid polymer stabilizer in water or aqueous solution.

In some embodiments, the dilution solution comprises about 10 to 100 mM Tris, about 10 to 200 mM boric acid, about 1 to 20 mM EDTA and optionally about 10 to 300 mM NaCl and/or about 0.1 to 0.5 wt % lipid polymer stabilizer in water or aqueous solution.

In some embodiments, the dilution solution comprises about 10 to 100 mM Tris, about 10 to 200 mM boric acid, about 1 to 20 mM EDTA, about 10 to 300 mM NaCl and about 0.1 to 0.5 wt % lipid polymer stabilizer in water or aqueous solution.

Without intending to be limited by theory, the boric acid in the dilution solution adjusts the pH of the biological sample. In certain embodiments, the dilution solution has a pH ranging from about 7 to 8.5 by employing TBE buffer based dilution solution. For example, the saliva dilution solution may have pH of about 7, of about 7.1, of about 7.2, of about 7.3, of about 7.4, of about 7.5, of about 7.6, of about 7.7, of about 7.8, of about 7.9, of about 8.0, of about 8.1, of about 8.2, of about 8.3, of about 8.0, or of about 8.5. Preferably, the saliva dilution solution has pH of about 8.0.

In some embodiments, the EDTA reduces mucin protein-protein interactions to reduce viscosity in biological samples like saliva.

In certain embodiments, the dilution solution may further comprise salts such as but not limited to sodium chloride, in a concentration of about 10 to 300 mM. For example, the concentration of sodium chloride in the saliva dilution buffer may be about 100 to 200 mM, or about 150 mM.

In certain embodiments, the dilution solution may further comprise a lipid polymer stabilizer in an amount of about 0.1 to 0.5 wt %, e.g., about 0.15 wt % to 0.20 wt %, based on the total weight of the dilution solution. In some embodiments, the lipid polymer stabilizer is a non-ionic surfactant including both hydrophobic groups (e.g., aliphatic group or fatty acid chain), hydrophilic groups (e.g., oxide or hydroxyl group), and/or amphiphilic groups. For example, the lipid polymer stabilizer may include polysorbate (e.g., Tween) or polyethylene glycol p-tert-octylphenyl ether (e.g., Triton), but examples are not limited thereto.

Accordingly, in some embodiments, the method of the present disclosure may further comprise a step of diluting the biological sample (e.g., saliva) using a diluting solution. The diluting solution may be utilized to optimize the biological sample in terms of its density, concentration, or fluidity for a desired sensitivity (e.g., change in optical properties). In some embodiments, the dilution solution inactivates the enzymes in samples such as saliva sample.

Thus, in some embodiments, the method of detecting presence or absence of a cannabinoid or metabolite thereof in a biological sample comprises:
diluting a biological sample in a dilution solution;
contacting the diluted biological sample with nanoparticle conjugated bioreceptor(s) capable of binding the cannabinoid or metabolite thereof, wherein
   a) the nanoparticle conjugated bioreceptor(s) are either immobilized on a fiber optic sensor, or
   b) the nanoparticle conjugated bioreceptor(s) are in solution phase; and
detecting a change in optical property of the fiber optic sensor in response to
a) direct binding of the cannabinoid or metabolite thereof to the nanoparticle conjugated bioreceptor(s) immobilized on the fiber optic sensor surface, or
b) binding of free nanoparticle conjugated bioreceptor(s) in solution phase to cannabinoid or metabolite thereof immobilized on a fiber optic sensor surface.

In some embodiments, the method of detecting presence or absence of a cannabinoid or metabolite thereof in a biological sample comprises:
diluting a biological sample in a dilution solution comprising one or more of salts, weak acids, weak bases, reducing agents, proteases, protease inhibitors and detergents;
contacting the diluted biological sample with nanoparticle conjugated bioreceptor(s) capable of binding the cannabinoid or metabolite thereof, wherein
   a) the nanoparticle conjugated bioreceptor(s) are either immobilized on a fiber optic sensor, or
   b) the nanoparticle conjugated bioreceptor(s) are in solution phase; and
detecting a change in optical property of the fiber optic sensor in response to
a) direct binding of the cannabinoid or metabolite thereof to the nanoparticle conjugated bioreceptor(s) immobilized on the fiber optic sensor surface, or
b) binding of free nanoparticle conjugated bioreceptor(s) in solution phase to cannabinoid or metabolite thereof immobilized on a fiber optic sensor surface.

In some embodiments, the method of detecting presence or absence of a cannabinoid or metabolite thereof in a biological sample comprises:
diluting a biological sample in a dilution solution comprising about 10 to 100 mM Tris, about 10 to 200 mM boric acid, and about 1 to 20 mM EDTA and optionally, about 10 to 300 mM salt and/or about 0.1 to 0.5 wt % lipid polymer stabilizer in water or aqueous solution;
contacting the diluted biological sample with nanoparticle conjugated bioreceptor(s) capable of binding the cannabinoid or metabolite thereof, wherein
   a) the nanoparticle conjugated bioreceptor(s) are either immobilized on a fiber optic sensor, or
   b) the nanoparticle conjugated bioreceptor(s) are in solution phase; and
detecting a change in optical property of the fiber optic sensor in response to
a) direct binding of the cannabinoid or metabolite thereof to the nanoparticle conjugated bioreceptor(s) immobilized on the fiber optic sensor surface, or
b) binding of free nanoparticle conjugated bioreceptor(s) in solution phase to cannabinoid or metabolite thereof immobilized on a fiber optic sensor surface.

In some embodiments, the ratio between the dilution solution and the biological sample ranges from about 1:1 to 1:10

The bioreceptor employed in the cannabinoid detection or detection device (e.g., biosensor device) is an agent capable of sensing, immobilizing or capturing cannabinoids or metabolites thereof to be measured and detected in a sample or recognizing and binding to antibodies directed against the cannabinoids or metabolites thereof. The bioreceptor includes, but is not particularly limited to, a peptide, a protein, antibody, an aptamer, a polymer, a hapten, a small molecule, a nucleic acid and an oligosaccharide, or any combination thereof.

In a preferred embodiment, the bioreceptor is an antibody capable of recognizing and binding to the cannabinoid or metabolite thereof in the optionally diluted biological sample.

In some embodiments, when the bioreceptor is an antibody, the antibody is selected from a group comprising antibodies capable of recognizing and binding THC, CBD and CBN or any combination thereof.

In a preferred embodiment, when the bioreceptor is an antibody, the antibody is an antibody capable of recognizing and binding THC, CBD and CBN or any combination thereof.

In some embodiments, the nanoparticle is preferably a metallic nanoparticle such as but not limited to a gold nanoparticle. The metallic nanoparticle, in some embodiments, may be formed in a layer or film.

In a preferred embodiment, the nanoparticle is a gold nanoparticle.

In another preferred embodiment, the bioreceptor(s) is conjugated to gold nanoparticle(s). Accordingly, in some embodiments, the nanoparticle conjugated bioreceptor(s) is a gold nanoparticle conjugated bioreceptor(s).

The size, geometry, etc. of the nanoparticle(s) may be optimized to improve the sensitivity of the biosensor device. For instance, various shapes of nanoparticles such as, round, cylindrical, rod shaped, etc. are within the scope of this disclosure.

In some embodiments, the size of the nanoparticles ranges from about 15 nm to about 60 nm.

In a preferred embodiment, the size of the nanoparticles ranges from about 15 nm to about 25 nm.

In yet another preferred embodiment, the size of the nanoparticles is about 20 nm.

In some embodiments, the nanoparticles may be pre-treated to contain a functional group that forms a covalent bond with the bioreceptor(s) on its surface. For example, the solid support may be treated or incubated with ethyl(dimethylaminopropyl) carbodiimide and N-hydroxysuccinimide, or hexamethylenediamine to form aldehyde or other functional groups on the surface of the solid support.

In some embodiments, when the biological sample is contacted with nanoparticle(s) conjugated bioreceptor(s) in solution phase, the fiber optic sensor is immobilized with the cannabinoid or metabolite thereof to be detected, wherein the cannabinoid or metabolite thereof is preferably conjugated to a carrier.

In the said variant of the method, the cannabinoid or metabolite thereof in the sample, if present, binds to nanoparticle(s) conjugated bioreceptor(s) in solution phase followed by contacting of the sample with the fiber optic sensor surface immobilized with cannabinoid/metabolite-carrier conjugate. In such a scenario, free or unbound nanoparticle conjugated bioreceptor(s) in solution phase bind to the cannabinoid/metabolite-carrier conjugate leading to a change in optical property of the fiber optic sensor. Thus, more the cannabinoid or metabolite thereof in the sample, lesser the amount of free or unbound nanoparticle conjugated bioreceptor(s) in solution phase, lower the binding of the free or unbound nanoparticle conjugated bioreceptor(s) to the fiber optic sensor and therefore, lower the change in optical property of the fiber optic sensor.

Accordingly, when the biological sample is contacted with nanoparticle(s) conjugated bioreceptor(s) in solution phase, change in optical property of the fiber optic sensor is detected in response to binding of free nanoparticle conjugated bioreceptor(s) in solution phase to cannabinoid or metabolite thereof immobilized on a fiber optic sensor surface in the form of a cannabinoid/metabolite-carrier conjugate.

In some embodiments, the carrier in the cannabinoid/metabolite-carrier conjugate may be selected from a group comprising Bovine Serum Albumin (BSA), serum globulins, albumins and ovalbumin or any combination thereof.

In some embodiments, the method of detecting presence or absence of a cannabinoid or metabolite thereof in a biological sample comprises:
contacting the biological sample with nanoparticle conjugated bioreceptor(s) capable of binding the cannabinoid or metabolite thereof, wherein
   a) the nanoparticle conjugated bioreceptor(s) are either immobilized on a fiber optic sensor, or
   b) the nanoparticle conjugated bioreceptor(s) are in solution phase; and
detecting a change in optical property of the fiber optic sensor in response to
a) direct binding of the cannabinoid or metabolite thereof to the nanoparticle conjugated bioreceptor(s) immobilized on the fiber optic sensor surface, or
b) binding of free nanoparticle conjugated bioreceptor(s) in solution phase to cannabinoid or metabolite thereof immobilized on a fiber optic sensor surface in the form of a cannabinoid/metabolite-carrier conjugate.

Thus, in some embodiments, the method of detecting presence or absence of a cannabinoid or metabolite thereof in a biological sample comprises:
diluting a biological sample in a dilution solution;
contacting the diluted biological sample with nanoparticle conjugated bioreceptor(s) capable of binding the cannabinoid or metabolite thereof, wherein
   a) the nanoparticle conjugated bioreceptor(s) are either immobilized on a fiber optic sensor, or
   b) the nanoparticle conjugated bioreceptor(s) are in solution phase; and
detecting a change in optical property of the fiber optic sensor in response to
a) direct binding of the cannabinoid or metabolite thereof to the nanoparticle conjugated bioreceptor(s) immobilized on the fiber optic sensor surface, or
b) binding of free nanoparticle conjugated bioreceptor(s) in solution phase to cannabinoid or metabolite thereof immobilized on a fiber optic sensor surface in the form of a cannabinoid/metabolite-carrier conjugate.

For further clarity, in a non-limiting example, said variant of the method wherein is contacted with bioreceptor(s) conjugated to nanoparticle(s) in solution phase, is based on a competitive plasmonic immunoassay realized using bioreceptor(s) conjugated to nanoparticle(s) (Eg. anti-THC antibody conjugated to nanoparticles (AuNPs)) in solution phase in combination with a fiber optic sensor immobilized with a conjugate comprising the cannabinoid or metabolite thereof to be detected and a carrier (Eg. BSA-THC). The competition occurs between the Δ9-THC molecules present in the biological (Eg. saliva samples) and the THC immobilized as part of the BSA-THC conjugate on to the probe surface of the fiber optic sensor. The binding of AuNPs to the fiber optic sensor surface may be quantified as loss of optical power intensity using a portable device fabricated as a part of this study, said aspect being further elaborated on in later sections of the present disclosure.

In some embodiments, the nanoparticle conjugated bioreceptor(s) or cannabinoid/metabolite-carrier conjugate(s) maybe immobilized on the surface of the fiber optic sensor directly or indirectly, in the form of a bioreceptor-nanoparticle conjugate.

Accordingly, in some embodiments, the nanoparticle(s) in the nanoparticle conjugated bioreceptor(s) is bound to the surface of the fiber optic sensor to immobilize the bioreceptor-nanoparticle conjugate to the said surface. In some embodiments, the nanoparticle is a metallic nanoparticle such as but not limited to a gold nanoparticle.

In some embodiments, the nanoparticle conjugated bioreceptor(s) may comprise one or more bioreceptor(s) bound to a single nanoparticle.

In a preferred embodiment, when the bioreceptor is an antibody, wherein the antibody is part of an antibody-nanoparticle conjugate, wherein the nanoparticle is bound to the surface of the fiber optic sensor to immobilize the antibody-nanoparticle conjugate to the said surface. In some embodiments, the said antibody-nanoparticle conjugate may comprise one or more antibodies bound to a single nanoparticle, wherein the nanoparticle is preferably a metallic nanoparticle such as but not limited to a gold nanoparticle.

In some embodiments, the probe region of the fiber optic sensor may be subjected to a pre-treatment to introduce a functional group that forms a covalent bond with the nanoparticle conjugated bioreceptor(s) or cannabinoid/metabolite-carrier conjugate(s). For example, the probe may be treated with ethyl(dimethylaminopropyl) carbodiimide and N-hydroxysuccinimide, or hexamethylenediamine to form aldehyde or other functional groups on the surface of the probe. In alternate embodiments of the present disclosure, the method may not require the pretreatment to introduce the functional group nor include formation of covalent bonding between the bioreceptors and the probe. For example, in some embodiments, the bioreceptors may associate with the probe only via physical attachment, engagement or absorption (“physisorption”). Alternatively, a portion of the bioreceptors may form chemical bonding with the probe while the rest of the bioreceptors may associate with the probe via physical attachment, engagement or absorption (“physisorption”).

In some embodiments, the method may, optionally, further include a step of incubating the biological sample and the nanoparticle conjugated bioreceptor(s) in a blocking agent.

The blocking agent is selected from a group comprising bovine serum albumin (BSA), casein, sericin, lipids, detergent, poly(ethylene glycol) and polyvinylpyrrolidone (PVP) or any combination thereof. In certain embodiments, the blocking agent preferably includes casein or sericin. In certain embodiments, the blocking agent preferably includes casein or sericin, but does not include the bovine serum albumin (BSA).

In a non-limiting embodiment, the step of contacting the biological sample with the nanoparticle conjugated bioreceptor(s) is performed for about 3 minutes to about 10 minutes.

In a preferred embodiment, the step of contacting the biological sample with the nanoparticle conjugated bioreceptor(s) may be performed for about 3 minutes to about 7 minutes.

In another preferred embodiment, the step of contacting the biological sample with the nanoparticle conjugated bioreceptor(s) may be performed for about 5 minutes.

In a non-limiting embodiment, the method of the present disclosure has a turnaround time of about 7 minutes to about 20 minutes.

In a preferred embodiment, the method of the present disclosure has a turnaround time of about 7 minutes to about 15 minutes.

In another preferred embodiment, the method of the present disclosure has a turnaround time of about 10 minutes.

In some embodiments, the above described method(s) of the present disclosure may be performed as a preliminary or confirmatory test to analyze the presence or absence of cannabinoid(s) or metabolite(s) thereof in a biological sample.

In some embodiments, the readout from the optical biosensor may be correlated with the amount of cannabinoid(s) or metabolite(s) thereof in a biological sample.

Accordingly, the above-described method(s) of the present disclosure may allow qualitative and/or quantitative analyses of a biological sample to confirm presence or absence of cannabinoid(s) or metabolite(s) thereof in a biological sample.

In some embodiments, the limit of detection of the above described method(s) of the present disclosure ranges from about 1 pg/mL to 100 ng/mL

Further details on the measurement of the change in optical properties of the optical biosensor in response to binding between the nanoparticle conjugated bioreceptor(s) and the cannabinoid or metabolite thereof are described in a subsequent section on the biosensor device per se.

Based on method outlined the above, the detection of the presence or absence of cannabinoids biomolecules or metabolites thereof in a sample using bioreceptors as envisaged in the present disclosure can thus be performed as
(a) a “label-free” technique or
(b) a “labeled” technique.

For further clarity, these variants of the method of the present disclosure have been further elaborated on below, wherein features relevant to the said variants touched upon in the above embodiments have not been repeated for reasons of brevity—

### “Label-Free” Technique

Envisaged in the present disclosure is a label-free technique for the detection of cannabinoid or metabolite thereof in a biological sample.

Accordingly, in some embodiments, the present disclosure provides a method of detecting presence or absence of a cannabinoid or metabolite thereof in a biological sample, comprising:
contacting a biological sample with nanoparticle conjugated bioreceptor(s) capable of binding the cannabinoid or metabolite thereof, wherein the nanoparticle conjugated bioreceptor(s) is immobilized on a fiber optic sensor, and
detecting a change in optical property of the fiber optic sensor in response to direct binding of the cannabinoid or metabolite thereof to the nanoparticle conjugated bioreceptor(s) immobilized on the fiber optic sensor surface.

The specifics of the above variant of the method in terms of the components involved, the time for which the biological sample is contacted with the bioreceptor, the turnaround time of the method, optional steps such as dilution of the sample, the ratio between the dilution solution and the sample, and pre-treatment of the probe region of the biosensor, unless otherwise described, are the same as the general method broadly described under the previous header and hence, the same has not been repeated herein, at least in entirety, for purposes of brevity.

In a preferred embodiment, the biological sample is saliva from a human subject.

In some embodiments, the biological sample is subjected to dilution before contacting with the bioreceptor.

The biological sample is subjected to dilution with a dilution solution comprising Tris, boric acid, EDTA and optionally salt and/or a lipid polymer stabilizer, in water.

The biological sample is subjected to dilution with a dilution solution comprising about 10 to 100 mM Tris, about 10 to 200 mM boric acid, and about 1 to 20 mM EDTA in water.

In some embodiments, the dilution solution comprises about 10 to 100 mM Tris, about 10 to 200 mM boric acid, about 1 to 20 mM EDTA, about 10 to 300 mM salt and about 0.1 to 0.5 wt % lipid polymer stabilizer in water or aqueous solution.

Thus, in some embodiments, the method of detecting presence or absence of a cannabinoid or metabolite thereof in a biological sample, comprises:
diluting a biological sample in a dilution solution comprising about 10 to 100 mM Tris, about 10 to 200 mM boric acid, about 1 to 20 mM EDTA, and optionally about 10 to 300 mM salt and about 0.1 to 0.5 wt % lipid polymer stabilizer in water or aqueous solution;
contacting the diluted biological sample with nanoparticle conjugated bioreceptor(s) capable of binding the cannabinoid or metabolite thereof, wherein the nanoparticle conjugated bioreceptor(s) is immobilized on a fiber optic sensor, and
detecting a change in optical property of the fiber optic sensor in response to direct binding of the cannabinoid or metabolite thereof to the nanoparticle conjugated bioreceptor(s) immobilized on the fiber optic sensor surface.

In some embodiments, the bioreceptor that is capable of binding the cannabinoid or metabolite thereof is selected from a group comprising a peptide, a protein, antibody, an aptamer, a polymer, a hapten, a small molecule, a nucleic acid and an oligosaccharide or any combination thereof.

In a preferred embodiment, the bioreceptor is an antibody capable of binding the cannabinoid or metabolite thereof.

In another preferred embodiment, the bioreceptor is an antibody against tetrahydrocannabinol (THC), cannabidiol (CBD) and cannabinol (CBN) or any combination thereof.

In yet another preferred embodiment, the bioreceptor is an antibody against THC or a derivative thereof.

In some embodiments, the nanoparticle is preferably a metallic nanoparticle such as but not limited to a gold nanoparticle.

Accordingly, in some embodiments, the method of detecting presence or absence of a cannabinoid or metabolite thereof in a biological sample, comprises:
contacting a biological sample with gold nanoparticle conjugated bioreceptor(s) capable of binding the cannabinoid or metabolite thereof, wherein the gold nanoparticle conjugated bioreceptor(s) is immobilized on a fiber optic sensor, and
detecting a change in optical property of the fiber optic sensor in response to direct binding of the cannabinoid or metabolite thereof to the gold nanoparticle conjugated bioreceptor(s) immobilized on the fiber optic sensor surface.

In some embodiments, the method of detecting presence or absence of a cannabinoid or metabolite thereof in a biological sample, comprises:
diluting a biological sample in a dilution solution comprising about 10 to 100 mM Tris, about 10 to 200 mM boric acid, about 1 to 20 mM EDTA, and optionally about 10 to 300 mM salt and about 0.1 to 0.5 wt % lipid polymer stabilizer in water or aqueous solution;
contacting the diluted biological sample with gold nanoparticle conjugated bioreceptor(s) capable of binding the cannabinoid or metabolite thereof, wherein the gold nanoparticle conjugated bioreceptor(s) is immobilized on a fiber optic sensor, and
detecting a change in optical property of the fiber optic sensor in response to direct binding of the cannabinoid or metabolite thereof to the gold nanoparticle conjugated bioreceptor(s) immobilized on the fiber optic sensor surface.

### “Labeled” Technique

In the context of labeled detection, the nanoparticle conjugated bioreceptor(s) is contacted with the optionally diluted biological sample in solution phase.

In a preferred embodiment, the bioreceptor(s) comprises an antibody against one or more of THC, CBD and CBN.

In another preferred embodiment, the bioreceptor(s) comprises an antibody against one or more of THC, CBD and CBN, wherein the bioreceptor is conjugated to metallic nanoparticle(s).

In yet another embodiment, the bioreceptor comprises an antibody against one or more of THC, CBD and CBN, wherein the bioreceptor(s) is conjugated to gold nanoparticle(s).

In some embodiments, the method of detecting presence or absence of a cannabinoid or metabolite thereof in a biological sample, comprises:
contacting the biological sample with nanoparticle conjugated bioreceptor(s) capable of binding the cannabinoid or metabolite thereof, wherein the nanoparticle conjugated bioreceptor(s) are in solution phase; and
detecting a change in optical property of the fiber optic sensor in response to binding of free nanoparticle conjugated bioreceptor(s) in solution phase to cannabinoid or metabolite thereof immobilized on a fiber optic sensor surface.

In some embodiments, the method of detecting presence or absence of a cannabinoid or metabolite thereof in a biological sample, comprises:
contacting the biological sample with gold nanoparticle conjugated bioreceptor(s) capable of binding the cannabinoid or metabolite thereof, wherein the gold nanoparticle conjugated bioreceptor(s) are in solution phase; and
detecting a change in optical property of the fiber optic sensor in response to binding of free gold nanoparticle conjugated bioreceptor(s) in solution phase to cannabinoid or metabolite thereof immobilized on a fiber optic sensor surface.

In some embodiments, when the biological sample is contacted with nanoparticle(s) conjugated bioreceptor(s) in solution phase, the fiber optic sensor is immobilized with the cannabinoid or metabolite thereof to be detected, wherein the cannabinoid or metabolite thereof is preferably conjugated to a carrier.

In the said variant of the method, the cannabinoid or metabolite thereof in the sample, if present, binds to nanoparticle(s) conjugated bioreceptor(s) in solution phase followed by contacting of the sample with the fiber optic sensor surface immobilized with cannabinoid/metabolite-carrier conjugate. In such a scenario, free or unbound nanoparticle conjugated bioreceptor(s) in solution phase bind to the cannabinoid/metabolite-carrier conjugate leading to a change in optical property of the fiber optic sensor. Thus, more the cannabinoid or metabolite thereof in the sample, lesser the amount of free or unbound nanoparticle conjugated bioreceptor(s) in solution phase, lower the binding of the free or unbound nanoparticle conjugated bioreceptor(s) to the fiber optic sensor and therefore, lower the change in optical property of the fiber optic sensor.

Accordingly, when the biological sample is contacted with nanoparticle(s) conjugated bioreceptor(s) in solution phase, change in optical property of the fiber optic sensor is detected in response to binding of free nanoparticle conjugated bioreceptor(s) in solution phase to cannabinoid or metabolite thereof immobilized on a fiber optic sensor surface in the form of a cannabinoid/metabolite-carrier conjugate.

In some embodiments, the carrier in the cannabinoid/metabolite-carrier conjugate may be selected from a group comprising Bovine Serum Albumin (BSA), serum globulins, albumins and ovalbumin or any combination thereof.

Accordingly, in some embodiments, the method of detecting presence or absence of a cannabinoid or metabolite thereof in a biological sample comprises:
contacting the biological sample with nanoparticle conjugated bioreceptor(s) capable of binding the cannabinoid or metabolite thereof, wherein the nanoparticle conjugated bioreceptor(s) are in solution phase; and
detecting a change in optical property of the fiber optic sensor in response to binding of free nanoparticle conjugated bioreceptor(s) in solution phase to cannabinoid or metabolite thereof immobilized on a fiber optic sensor surface in the form of a cannabinoid/metabolite-carrier conjugate.

In some embodiments, the method of detecting presence or absence of a cannabinoid or metabolite thereof in a biological sample comprises:
diluting the biological sample in a dilution solution;
contacting the diluted biological sample with nanoparticle conjugated bioreceptor(s) capable of binding the cannabinoid or metabolite thereof, wherein the nanoparticle conjugated bioreceptor(s) are in solution phase; and
detecting a change in optical property of the fiber optic sensor in response to binding of free nanoparticle conjugated bioreceptor(s) in solution phase to cannabinoid or metabolite thereof immobilized on a fiber optic sensor surface in the form of a cannabinoid/metabolite-carrier conjugate.

In some embodiments, the method of detecting presence or absence of a cannabinoid or metabolite thereof in a biological sample comprises:
diluting a biological sample in a dilution solution comprising about 10 to 100 mM Tris, about 10 to 200 mM boric acid, and about 1 to 20 mM EDTA and optionally, about 10 to 300 mM salt and/or about 0.1 to 0.5 wt % lipid polymer stabilizer in water or aqueous solution;
contacting the diluted biological sample with nanoparticle conjugated bioreceptor(s) capable of binding the cannabinoid or metabolite thereof, wherein the nanoparticle conjugated bioreceptor(s) are in solution phase; and
detecting a change in optical property of the fiber optic sensor in response to binding of free nanoparticle conjugated bioreceptor(s) in solution phase to cannabinoid or metabolite thereof immobilized on a fiber optic sensor surface in the form of a cannabinoid/metabolite-carrier conjugate.

As mentioned above, in some embodiments, said variant of the method is based on a competitive plasmonic immunoassay realized using bioreceptor(s) conjugated to nanoparticle(s) (Eg. anti-THC antibody conjugated to gold nanoparticles (AuNPs)) in solution phase in combination with a fiber optic sensor immobilized with a conjugate comprising the cannabinoid or metabolite thereof to be detected and a carrier (Eg. BSA-THC). The competition occurs between the Δ9-THC molecules present in the biological (Eg. saliva samples) and the THC immobilized as part of the BSA-THC conjugate on to the probe surface of the fiber optic sensor. The binding of AuNPs to the fiber optic sensor surface may be quantified as loss of optical power intensity using a portable device fabricated as a part of this study, said aspect being further elaborated on in later sections of the present disclosure.

### Fiber Optic Biosensor Device

In some embodiments, the change in optical property of the fiber optic sensor is measured using a device comprising:
i) a light source located proximal to one end of the optical fiber; and
ii) a detector located proximal to another end of the optical fiber, wherein the detector is configured to sense the change in the optical property of light that traverses through the optical fiber when the probe region is exposed to the biological sample comprising the cannabinoid or metabolite thereof.

For example, the fiber optic biosensor device may include a light source located proximate to one end of an optical fiber for illuminating the optical fiber at a predetermined wavelength. The optical fiber, in some non-limiting embodiments, may be bent to form a U-shape such that light from the light source may traverse the U-bent region of the optical fiber before being received by a detector located proximate to another end of the optical fiber. A portion of the optical fiber may be a probe region that is functionalized (i.e., bioreceptor molecules or the cannabinoid/metabolite-carrier conjugate) for interaction with optionally pre-treated biological sample and/or target biomolecules (described below) where any such interaction causes a change in the properties of light traversing the optical fiber and can be detected by the detector.

The light source may include, for example, one or more light emitting diodes (LEDs) configured to emit light in the visible, infrared (IR), and/or ultraviolet (UV) wavelengths. In some embodiments, a green, red, yellow, or another color LED may be used. Light emitted by the light source may, optionally, be controlled using a feedback circuit based on signal or information received by the detector. For example, current driven to the light source may be maintained at the desired level.

The detector may correspondingly operate in the visible, infrared (IR), and/or ultraviolet (UV) wavelengths. Examples of the detector may include, without limitation, a spectrometer (spectrophotometer, spectrograph, spectroscope, or other suitable device), an optical detector sensitive at a particular localized surface plasmon resonance (LSPR) frequency, photodiode, phototransistor, or the like.

The probe region of the optical fiber comes in contact with the sample suspected of comprising the target biomolecules or analyte. Typically, the complete probe region or a portion thereof may come in contact with the target biomolecules or analyte (i.e. cannabinoid or metabolite thereof).

In some embodiments, the probe region in a portion of the optical fiber may be coated with suitable bioreceptor molecules or the cannabinoid/metabolite-carrier conjugate (directly or indirectly), wherein before application of the said coating, the probe may optionally be functionalized or biofunctionalized to facilitate efficient coating or immobilization of the said molecules. The coating facilitates the binding of the target biomolecules or analytes to the surface of the probe region. The bioreceptor molecules or the cannabinoid/metabolite-carrier conjugates may include any of those as described in the earlier embodiments, without any limitation.

In some embodiments, the probe region may optionally comprise an uncladded exposed fiber core with metallic nanoparticle coating on the uncladded exposed fiber core.

When a molecule is bound (e.g., immobilized by functionalization), to an uncladded portion of the optical fiber, it may absorb the evanescent wave, and the measured absorption spectra may be used to detect presence, concentration and/or other properties of the molecule, as explained in more detail below. This is called evanescent wave absorbance phenomenon.

The term “evanescent waves”, in the context of the optics used in this disclosure, refer to waves that are formed at the core/cladding interface as the light passing through the fiber core undergoes total internal reflection. In all optical fibers, light propagates by means of total internal reflection, wherein the propagating light is launched into waveguide at angles such that upon reaching the cladding-core interface, the energy is reflected and remains in the core of the fiber. For light reflecting at angles near the critical angle, a significant portion of the power extends into the cladding or medium which surrounds the core. This phenomenon, known as the evanescent wave (EW), extends only to a short distance from the interface, with power dropping exponentially within a distance of λ/10 (typically less than 50 nm) from the core/cladding interface. This is called an evanescent field or evanescent wave (EW). Cladding material (either polymer or silica) is generally removed in order to access these evanescent waves. The cladding material may be removed, for example, by using a sharp surgical blade, a file, sand paper, or any other abrasive tool.

Evanescent wave-based absorbance sensitivity of bare (unclad, uncoated surface of fiber core) fiber optic probes can be increased by modifying the probe geometry.

Different optical fiber designs including straight, U-bent, tapered tip, and biconical tapers may be employed in the development of absorbance based bio/chemical sensors. Some embodiments relate to U-bent optical fibers having one or more of (and not limited to) good sensitivity, compactness, ease in fabrication, and possibly higher compatibility with instrument configurations. Without intending to be limited by theory, evanescent fields around U-bent probes are stronger than in a straight probe.

In some embodiments, at least a part of the U-bent region may form the probe region. In some embodiments, an uncladded portion of the U-bent region (e.g., the tip) may form the probe region. Optionally, at least a part of the straight arm forms the probe region. At the U-bent region, light interacts with biomolecules bound to the bioreceptor or to the conjugate comprising the cannabinoid or metabolite thereof to be detected and carrier and traverses through the second arm of the optical fiber towards the detector end. For example, an interaction with a target biomolecule may lead to modulation in the effective refractive index (RI) and/or evanescent wave absorbance of light waves traversing through the functionalized portion as a function of target biomolecule type, concentration, and/or other properties. The interaction signal may be amplified by coating the probe region 105 with gold nanoparticles (or other metallic film) that may, for example, lead to greater loss in the light and may be measured usually as an increase in the absorbance value (or decrease in the intensity count). Immobilization of bioreceptors or the conjugate comprising the cannabinoid or metabolite thereof to be detected and carrier and immunocomplex formation during biomolecule binding on the surface of gold nanoparticles leads to increase in effective refractive index of the microenvironment causing a change in properties of light traversing through the optical fiber. Further, the application of U-bent fiber facilitates the placement of light source and detector and eliminates the use of additional optical components including beam splitters.

Although the length of biosensor probe and the resolution of a biosensor are directly proportional, the length of the probe cannot be increased beyond a certain limit as the sample analyte volume required also increases. The biosensors of the embodiments herein, however maybe length independent because U-bent optical fibers have increased sensitivity and could be configured for use with low sample volume of 100 microliters or less. However, if a larger or wider flowcell is used, a larger sample volume can be accommodated. Indeed, the biosensor can be used without a flow cell—the biosensor can work in any body of analyte, for example, a beaker, cup, glass, pond or river. The embodiments herein satisfy the need for a biosensor with increased sensitivity and/or resolution irrespective of the length.

In certain embodiments, surface plasmon resonance (SPR) may be used to further increase sensitivity and/or accuracy of detection by coating the surface of the optical fiber under cladding with a thin film of metal such as silver or gold nanoparticles that act as substrate for immobilization of the bioreceptor molecules or the cannabinoid/metabolite-carrier conjugate. A surface plasmon is an oscillation of free electrons that propagates along the surface of a conductor. The phenomenon of SPR occurs under total internal reflection conditions at the boundary between substances of different refractive indices, such as glass and water solutions. When an incident light beam is reflected internally within the first medium, its electromagnetic field produces an evanescent wave that crosses a short distance (in the order of nanometers) beyond the interface with the second medium. If a thin metal film is inserted at the interface between the two media, surface plasmon resonance occurs when the free electron clouds in the metal layer (the plasmons) absorb energy from the evanescent wave and cause a measurable drop in the intensity of the reflected light at a particular angle of incidence that depends on the refractive index of the second medium. Surface plasmon resonance reflectivity measurements, as discussed in more detail below, can be used to detect molecular adsorption, such as by cannabinoid biomolecules in a sample.

Typically, the conductor used for SPR spectrometry is a thin film of metal such as silver or gold; however, surface plasmons have also been excited on semiconductors, noble-metal containing nanoparticle includes one or more of rhodium, iridium, palladium, silver, osmium, iridium, platinum, gold or combinations thereof, or any material which exhibits surface plasmon resonance (e.g., copper and aluminum). The conventional method of exciting surface plasmons is to couple the transverse-magnetic (TM) polarized energy contained in an evanescent field to the plasmon mode on a metal film. The amount of coupling, and thus the intensity of the plasmon, is determined by the incident angle of the light beam and is directly affected by the refractive indices of the materials on both sides of the metal film. By including the sample material to be measured as a layer on one side of the metallic film, changes in the refractive index of the sample material can be monitored by measuring changes in the surface plasmon coupling efficiency in the evanescent field. When changes occur in the refractive index of the sample material, the propagation of the evanescent wave and the angle of incidence producing resonance are affected. Therefore, by monitoring the angle of incidence at a given wavelength and identifying changes in the angle that causes resonance, corresponding changes in the refractive index and related properties of the material can be readily detected.

Nanoparticles of noble metals such as gold and silver are known to exhibit optical absorption and scattering properties in UV (approximately 10-380 nm)-visible (approximately 380-760 nm)-near IR region (Approximately (760-2,500 nm) termed as localized surface plasmon resonance (LSPR). The extinction band due to LSPR can be influenced by the size, shape and composition of nanoparticles and most importantly by the surrounding environment. Refractive index changes taking place at the surface of the nanoparticles result in changes in absorbance and a red-shift in absorbance peak (λmax). The LSPR properties of gold and silver nanoparticles can be utilized in liquid phase as well as in monolayers coated on glass/quartz substrates, for example, for detecting cannabinoids intake or consumption. One embodiment may include gold capped silica/polystyrene nanoparticle coated substrates. Other embodiments may include nanoparticles of rhodium, iridium, palladium, silver, osmium, iridium, platinum, or combinations thereof. The absorbance response of MNPs-based sensors can be further enhanced by coating MNPs on an efficient absorbance based sensor, such as a siloxane polymer. Sensitivity of optical fiber probes can be enhanced using the LSPR-based biosensor by using a fiber optic evanescent-wave sensing scheme. MNP coated on uncladded straight fiber probes can be used for chemical and biochemical sensing. In an alternative embodiment, the MNPs may be coated on a bent optical fiber. Various shapes of nanoparticles such as, round, cylindrical, rod shaped, etc. are within the scope of this disclosure. In some embodiments, the size of the nanoparticles (e.g., gold nanoparticles) may be about 15 nm to about 60 nm.

It should be noted that for the label-free sensing approach described below, gold nanoparticles may be used because the exhibit LSPR. It can also be used for labeled sensing approaches to further increase sensitivity. It should be noted that LSPR eliminates the need for the use of polarized light. Similarly, use of LSPR eliminates the angle of incidence as a constraint for sensing.

Immobilization of one or more bioreceptors or conjugate comprising the bioreceptor molecules or the cannabinoid/metabolite-carrier conjugate onto a probe region of the optical biosensor may be performed so that it may not be washed away by rinsing procedures, and/or its binding to target biomolecules test sample is unimpeded by the probe surface. One or more specific bioreceptors or conjugates comprising the cannabinoid or metabolite thereof to be detected and carrier can be attached to a probe surface by physical adsorption (i.e., without the use of chemical linkers) or by chemical binding (i.e., with the use of chemical linkers). Chemical binding can generate stronger attachment of specific binding substances on a probe surface and provide defined orientation and conformation of the surface-bound bioreceptor molecules or the cannabinoid/metabolite-carrier conjugate. Examples of chemical binding include, for example, amine activation, thiol-PEG-NHS activation, aldehyde activation, and nickel activation. In some embodiments, bioreceptor molecules or the cannabinoid/metabolite-carrier conjugate may be labeled to enhance the detection signal.

In some embodiments, the bioreceptor molecules or the cannabinoid/metabolite-carrier conjugates are covalently bound to the surface of the optical biosensor. The said covalent binding, in some embodiments, is achieved by pre-treating the probe to introduce a functional group that forms a covalent bond with the bioreceptor molecules or the cannabinoid/metabolite-carrier conjugate on the surface of the probe.

Thus, in some embodiments, the fabrication of the optical fiber for its use in the method of the present disclosure, includes a step of pre-treating the probe to introduce a functional group that forms a covalent bond with the bioreceptor on the surface of the probe. In some non-limiting embodiments, the probe region of the optical fiber is treated or incubated with ethyl(dimethylaminopropyl) carbodiimide and N-hydroxysuccinimide, or hexamethylenediamine to form aldehyde or other functional groups on the surface of the probe.

In some embodiments, the fiber probes may be functionalized with silanol groups, for examples using mercaptosilane.

In certain embodiments, the method may not require the pretreatment to introduce the functional group nor include formation of covalent bonding between the bioreceptor molecules or the cannabinoid/metabolite-carrier conjugate and the probes. For example, the bioreceptor molecules or the cannabinoid/metabolite-carrier conjugate may associate with the probes only via physical attachment, engagement or absorption (“physisorption”). Alternatively, a portion of the bioreceptor molecules or the cannabinoid/metabolite-carrier conjugate may form chemical bonding with the probes while the rest of the bioreceptors or conjugates may associate with the probes via physical attachment, engagement or absorption (“physisorption”).

The detector may sense optical power loss in the light or change in the absorbance/intensity count propagating in the fiber-optic probe due to the presence of a target biomolecule bound to the probe region. Any specific interaction between the bioreceptor or the conjugate comprising the cannabinoid or metabolite thereof to be detected and carrier and the biomolecule of interest leads to modulation in the effective refractive index (RI) and/or evanescent wave absorbance as a function of biomolecule concentration, and can be used for qualitatively and quantitatively calculating measured values of target biomolecules (e.g., concentration, presence/absence, etc.). The use of gold nanoparticles further amplifies the interaction signal that eventually leads to greater loss in the light and is measured usually as an increase in the absorbance value (or decrease in the intensity count). When a biological sample including a target biomolecule is supplied to the bioreceptor, the detector may sense optical power loss generated either by labeling the bioreceptor with a specific label (i.e., in line with the labeled variant of the method of the present disclosure) capable of generating optical power loss signals or by further adding a substance capable of generating optical power loss signals without labeling (i.e., in line with the label-free variant of the method of the present disclosure), and represents qualitatively and quantitatively measured values such as constituents, presence or absence and concentrations of the biological samples.

The concentration of a target biomolecule in a biological sample may be determined based on, for example, the radius of the optical fiber into which light is coupled, the numerical aperture at the sensing region of the fiber, the wavelength of light, the extinction coefficient of absorbing medium (GNP), and the concentration of absorbent molecules (i.e., bioreceptor) bound per unit circumferential surface area of the fiber, or the like. For example, the concentration may be proportional to the extinction coefficient, the wavelength of light at which the extinction is maximum (or a threshold), and/or the number of nanoparticles on the probe surface.

The detector may further convert the optical signal to an electrical signal using any now or hereafter known techniques. The electrical signal may be transmitted to a processor of the biosensor device and/or an external device for processing (i.e., analysis) such as determination of concentration, detecting presence or absence, and/or other properties of a target biomolecule. In one embodiment, it is possible to simplify the readout instrumentation by the application of a filter 108 so that only positive results over a determined threshold trigger a detection.

The biosensor device may, optionally, include one or more of a processor configured for, for example, analyzing the detected signal and generating an output; a controller configured for, for example, controlling or programming the functionality of the biosensor device, including the light source, the detector and/or the optical fiber; a display configured for, for example, displaying instructions, results, etc.; a power source (e.g., via a USB connection); and/or other components (e.g., detector signal filters, etc.). One or more of the components may be included in the sensor device and/or may be located externally and in communication with the sensor device via an, optional, communications interface.

Furthermore, while not shown here, a sample holder may hold a biological sample from a subject and may be brought in contact with the probe region of the optical biosensor device. For example, in an embodiment, the probe region of the biosensor device may be inserted into the sample holder such that it contacts the biological sample contained within. The sample holder may take a variety of configurations and in some embodiments the sample holder may be in the form of a cartridge, a cuvette, a test tube, a fluidic channel, a petri dish, a microtiter plate well, or the like. Optionally, an identifier (ID) detector may detect an identifier on the sample holder. The identifier detector may communicate may transmits the identifier to an external device (or a controller). Where desired, the external device and/or controller may identify a protocol to be run on the sample holder that may comprise instructions to the controller of the reader assembly to perform the protocol on the sample holder, including but not limited to a particular assay to be run and a detection method to be performed. Once the assay is performed on the sample holder, a signal indicative of cannabinoid or metabolite thereof in the biological sample is generated and detected by the detector.

To ensure that a given sensor response (e.g. a light intensity) correlates with an accurate concentration of a target biomolecule of interest in a biological sample, the biosensor device may be calibrated before detecting the response using any now or hereafter known calibration protocols. For example, a control assay (e.g., control probe or nanoparticles) to detect another type of biomolecule can be used in the same sample as a calibrator or control. Alternatively, a second probe may be dipped into the sample at the same time for calibrating purpose.

In some embodiments, a variant probe that is biofunctionalized to detect multiple biomolecules and the sample mixture may include various sets of nanoparticles having different affinity, shape or size.

### Kits

In some embodiments, further provided herein is a test kit for detecting presence or absence of a cannabinoid or a metabolite thereof in a biological sample.

In some embodiments, the kit comprises the biosensor as described in the above embodiments.

In some embodiments, the kit may further comprise means for sample collection and optionally, quantification and/or storage.

In some embodiments, examples of the means for sample collection include but are not limited to a sponge, swab material, such as cotton or synthetic fiber. Once collected, the biological sample from the swab may be extracted by an external mechanism or may be directly applied to the biosensor device.

In some embodiments, the means for sample collection and storage may be the same. Examples of such means for sample collection and/or storage include but are not limited to a tube, cylinder and sponge. Types of such means for sample collection and/or storage are not particularly limited. In certain embodiments, the kit may further include a device for measuring, e.g., measuring volume of sample, for sufficient dilution suitable for automatic measuring or detection.

In some embodiments, the test kit may further comprise a vial or container comprising the blocking agent as described in the previous sections of the present disclosure.

In some embodiments, the test kit may further comprise a second vial or container comprising the sample diluting solution as described in the previous sections of the present disclosure or components thereof, wherein the diluting solution may be utilized to optimize the biological sample in its density, concentration, or fluidity for a desired sensitivity (e.g., change in optical properties) in the biosensor device.

Accordingly, envisaged herein is a test kit for detecting a cannabinoid or metabolite thereof in a biological sample, comprising:
the fiber optic biosensor device is as described above;
means for sample collection and optionally, quantification and/or storage;
and one or more of:
a vial or container comprising a blocking agent;
a second vial or container comprising a sample diluting solution or components thereof;
and
an instruction manual.

In some embodiments, the test kit may further include a sample collection and metering device through which the sample is collected, automatically processed, diluted, and mixed with the nanoparticle conjugated bioreceptor to optimize the biological sample in its density, concentration and/or fluidity for desired sensitivity in the biosensor device.

### Applications

The present disclosure further provides use of the fiber optic biosensor device or the test kit as described above for the qualitative and/or quantitative detection of cannabinoid or metabolite thereof in a biological sample.

### EXAMPLE

### Example 1: Competitive Immunoassay

### Materials

Silica fibers (FT200UMT, core diameter: about 200 μm) were procured from Thorlabs Inc., USA or CeramOptec, Germany. The bioreagents such as tetrahydrocannabinol-bovine serum albumin conjugate, monoclonal mouse anti-tetrahydrocannabinol antibody were purchased from Fitzgerald, USA, and Novus Biologicals, USA respectively. Other bioreagents such as human immunoglobulin G (HIgG) and goat anti-human immunoglobulin G (GaHIgG) were obtained from Sigma Aldrich. Gold nanoparticles (AuNPs, OD=1) of varying sizes such as about 20 nm, about 30 nm, about 40 nm, and about 60 nm procured from BBI Solutions, UK or nanoComposix, USA. Other chemicals obtained were of analytical grade.

### Saliva and Saliva Dilution Solution

All experiments were carried out using artificial saliva.

A saliva dilution solution comprising about 100 mM Tris, about 90 mM boric acid, about 10 mM EDTA, about 150 mM NaCl, and about 0.15% lipid polymer stabilizer in water was prepared. It was found that this combination of reagents produced better results for both buffering the pH (to about pH 8) and viscosity to reduce variability between individual subjects compared to other tested buffers (e.g., including Tris-HCl, PBS, etc.).

### Fabrication and Functionalization of U-Bent Fiber Optic Sensor Probe

The U-bent silica fiber probes were cleaned by sonication in acetone (about 15 min, about 1000 Watt, about 28 kHz). The cleaned U-bent regions (referred to as sensor) of the fiber probes were treated with piranha solution for about 20 min at a temperature of about 60° C. to oxidize organic contaminations and generate silanol groups on the sensor surface. After that, the fiber probes were washed with deionized water and dehydrated for about 1 h at a temperature of about 115° C. to remove the physisorbed water.

For amino-silanization, the fiber probes were dipped in a about 1% solution of APTMS in about a 5:2 (v/v) mixture of ethanol and acetic acid (about 5 min). Finally, the fiber probes were washed thrice in ethanol, sonicated (about 15 min), and dried at a temperature of about 100° C. for about 1 hour. The salinized sensor probes were dipped into about 1% glutaraldehyde (about 500 μL, about 30 min, room temperature) and washed in DI water (3×100 μL, about 3 min). Then after, the aldehyde-functionalized sensor fiber probes were incubated in the protein solution. Firstly, to obtain a proof-of-concept on competitive fiber optic immunoassay, HIgG and GaHIgG were utilized. For this purpose, the aldehyde treated fiber probes were dipped in about 50 μL of about 50 μg/mL HIgG overnight at a temperature of about 4° C.

Then, to demonstrate the detection of THC the aldehyde treated fiber probes were dipped in about 50 μL of about 50 μg/mL BSA-THC solution each (overnight, about 4° C.). The next day the samples were dipped into PBST solution (100 μL, about 2 min) to remove loosely bound antibodies.

### Preparation of Solution Phase Antibody Bioreceptor Conjugates

Briefly, 1 ml solution of AuNP (OD 1, about pH 8.5) was added with about 100 μl of about 25 μg/ml of GaHIgG or anti-THC antibody, and incubated for 15 mins at RT. Soon after, about 80 μl of about 320 μM SH-PEG was added and incubated for about 15 mins. Then, the reaction mixture was centrifuged at about 7000 RPM for about 25 mins at about 4° C. to remove unbound antibodies. The supernatant was discarded and the AuNP labels were resuspended using about 1 ml of PBS (1×, about pH 7.4) centrifugation step was repeated thrice. After, three rounds of centrifugation the AuNP labels were diluted to various concentrations such as 1×, 5×, and 10× using PBS solution.

### Optical Setup

The U-bent sensor probe with the corresponding antibody was connected between narrowband green LED (LED528EHP, about 7 mW, Thorlabs Inc. USA) driven by a constant current circuit and a photodetector (about 10 pW-50 mW, S150C, Thorlabs Inc., USA). The LED was housed in a custom-made unit with a SMA holder. The photodetector was connected to a computer through a USB console PM100USB (Thorlabs Inc., USA). The optical intensity changes at the photodetector were monitored in real-time by choosing the specific wavelength depending on the size of the AuNP labels with an integration time of about 0.1 microsecond averaged over 3000 consecutive measurements to improve the signal to noise ratio. All experiments were carried out at room temperature (about 25° C.). The intensity responses were converted into corresponding absorbance values for further analysis (mainly to appreciate small changes in the intensity).

### Results

### Competitive Fiber Optic Immunoassay

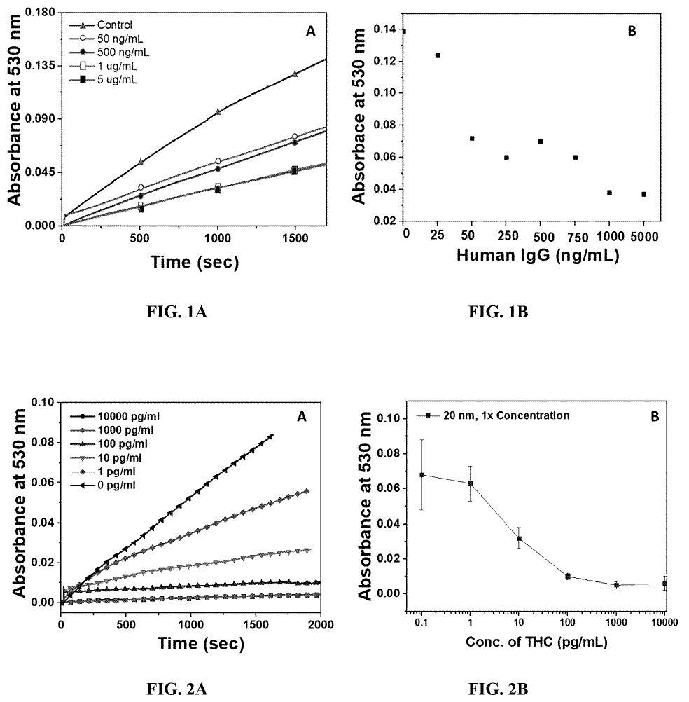

For a competitive immunoassay with the U-bent fiber probe sensor, HIgG was utilized as a model analyte molecule. Briefly, HIgG was immobilized on to the fiber sensor surface, while GaHIgG was conjugated to with AuNPs. Firstly, a plasmonic immunocomplex was prepared by mixing and incubating (about 5 mins, at about room temperature (RT)) about 30 μL of AuNP labels and 30 μL of varying concentrations of HIgG ranging from 0 ng/ml to 5000 ng/ml prepared in PBS. Then, the sensor probe is subjected to plasmonic immunocomplex, while the drop in intensity is monitored and recorded. In case of AuNP labels mixed with 0 ng/ml of HIgG, the nanoparticle conjugated antibody binds freely with the HIgG on surface without any hindrance. While, in presence of HIgG in immunocomplex mixture the GaHIgG on the surface gets passivated with the HIgG in solution, hence inhibiting the binding of nanoparticle conjugated antibody to the sensor probe (FIGS. 1A, 1B). Theoretically, a complete inhibition of nanoparticle conjugated antibody is expected, when the entire antibodies on the AuNP surface gets occupied or passivated with HIgG from the sample solution.

As shown in FIGS. 1A-1B, competition between the HIgG in the sample solution and the HIgG on the sensor probe surface is shown. The concentration of about 1 ug/ml or less was enough to completely inhibit the binding of nanoparticle conjugated antibody to the sensor surface.

### Competitive Immunoassay for the Detection of THC: Chemisorption of BSA-THC

The aldehyde treated fiber probes were immobilized with BSA-THC and the plasmonic labels of 20 nm AuNP were made by conjugating with anti-THC antibody.”

Briefly, BSA-THC was immobilized on to the fiber sensor surface, while anti-THC antibody was conjugated to AuNPs to form plasmonic labels. Firstly, the THC sample (30 μL, in PBS buffer) was added to AuNP labels (20 nm, 30 μL, 10× concentration) and mixed thoroughly by pipetting the mixture for κ times and incubating the mixture for 5 min to form a plasmonic immunocomplex (AuNP-IgG-THC). Then, the sensor probe covalently immobilized with BSA-THC was dipped in this mixture to allow the binding of AuNP-IgG plasmonic labels with vacant THC binding sites to the BSA-THC on the probe surface. The optical intensity changes were monitored in real-time, due to absorption of the light passing through the probe caused by the plasmonic labels bound to the probe surface. The Studies were performed for various concentrations of THC from 0 to 10,000 μg/mL. When the sample has low concentration of THC, more AuNP-IgG labels will be free to bind to the probe surface. Hence, the absorbance response is inversely proportional to the THC conc in the sample (FIG. 2A-2B).

Since the lower cut-off for THC concentration was 5 ng/mL, which means that THC has to be detected above these concentrations only, numerical calculations were performed to understand the effect of AuNP size (FIG. 3).

### Competitive Immunoassay for the Detection of THC: Chemisorption of BSA-THC

The aldehyde treated fiber probes were immobilized with BSA-THC and the nanoparticle conjugated antibody having 20 nm AuNP were made by conjugating with anti-THC antibody. Following which, the biosensor probe was challenged with varying concentration of THC.

TABLE 1
Absorbance response due to binding of nanoparticle conjugated
antibody by competitive fiber optic immunoassay

| THC Conc. | Absorbance | Std. dev. |
|---|---|---|
| (pg/ml) | response | n = 3 |
| 0 | 0.075 | 0.02 |
| 0.1 | 0.068 | 0.02 |
| 1 | 0.063 | 0.01 |
| 10 | 0.032 | 0.006 |
| 100 | 0.01 | 0.002 |
| 1000 | 0.005 | 0.002 |

Table 1 above shows the absorbance response due to binding of nanoparticle conjugated antibody by competitive fiber optic immunoassay. A higher absorbance response is seen in case of lower THC concentration due to lack of competitive binding of THC with the anti-THC antibody in the sample.

The results show that a concentration of about 100 μg/ml is enough to saturate the tested nanoparticle conjugated antibody of 20 nm. However, depending on the application it may be necessary to shift the active dynamic range to ng/ml. To achieve the same, a theoretical simulation was performed considering varying size and concentration of AuNP labels. Three sizes of AuNPs such as 20 nm, 30 nm and 40 nm at concentrations 1×, 5× and 10× (Table 2) were considered and the simulation was carried out assuming each 20 nm AuNP label contains one active antibody, while each 30 nm AuNP labels contain two active antibodies and each 40 nm AuNP labels contain at least 10 active antibodies. Thus, based on the number of THC present in about 30 μl of the sample solution, the number AuNP coming to the surface varies as shown in FIG. 3. The simulated results showed that the dynamic range could be varied depending on the size and concentration of AuNP labels.

TABLE 2
No. of AuNPs present in 30 μl of AuNP solution

| | | Optical | | | |
|---|---|---|---|---|---|
| S. | AuNP | density | No. of AuNPs in 30 μl of OD 1 solution | | |
| No | size | (OD) | Conc. 1× | Conc. 5× | Conc. 10× |
| 1 | 20 nm | 1 | 2*10¹⁰ | 10*10¹⁰ | 21*10¹⁰ |
| 2 | 30 nm | 1 | 6*10⁹ | 3*10¹⁰ | 6*10¹⁰ |

TABLE 3
Absorbance response obtained using AuNP
labels of varying size and concentration

| | | | Control | 5 ng/ml |
|---|---|---|---|---|
| | | | Absorbance | Absorbance |
| Sample | AuNP | | response | response |
| No. | size | Concentration | (0 ng/ml) (n = 3) | (n = 3) |
| 1 | 20 nm | 1× | 0.009 ± 0.002 | |
| | | | (in 3 mins) | |
| | | 5× | 0.011 ± 0.006 | 0.004 ± 0.002 |
| | | | (in 3 mins) | (in 3 mins) |
| | | | 0.03 ± 0.009 | |
| | | | (in 3 mins) | |
| | | 10× | 0.04 ± 0.012 | 0.005 ± 0.002 |
| | | | (in 3 mins) | (in 3 mins) |
| | | | 0.089 ± 0.01 | |
| | | | (in 10 mins) | |
| 2 | 30 nm | 5× | 0.033 ± 0.01 | 0.004 ± 0.003 |
| | | | (in 3 mins) | (in 3 mins) |
| | | 10× | 0.021 ± 0.009 | 0.005 ± 0.002 |
| | | | (in 3 mins) | (in 3 mins) |
| 3 | 40 nm | 5× | 0.03 ± 0.001 | 0.009 ± 0.003 |
| | | | (in 3 mins) | (in 3 mins) |
| | | 10× | 0.028 ± 0.003 | 0.007 ± 0.003 |
| | | | (in 3 mins) | (in 3 mins) |

Table 3 shows the absorbance response obtained using AuNP labels of varying size and concentration with respect the considered THC concentration (n=3). With 30 and 40 nm AuNPs the results were not consistent, a drastic drop in the intensity (both with control and 5 ng/ml) was observed as soon as the sensor probe is transferred from PBS to conjugate solution, which is different from that of the kinetics observed with 20 nm AuNPs.

It could be noted from the experiments that the absorbance response does not follow the simulated results, as the 30 and 40 nm AuNPs repeatedly resulted in lesser response and 20 nm at 10× concentration seems to be optimum. On further analysis of the data it was found that the BSA was conjugated to THC using amine-carboxylic binding. Here, to immobilize the BSA-THC same chemistry was explored, hence if the amine groups in the BSA are blocked then it may not bind to the fiber probes. To validate the hypothesis experiments were carried out by physisorption of BSA-THC on to the sensor surface.

### Competitive Immunoassay for the Detection of THC: Physisorption of BSA-THC

The cleaned U-bent sensor probes without any surface functionalization were dipped in BSA-THC overnight. Then, the BSA-THC immobilized sensor probes were utilized of the competitive fiber optic immunoassay using 10× concentration of 20, 30 and 40 nm AuNP labels.

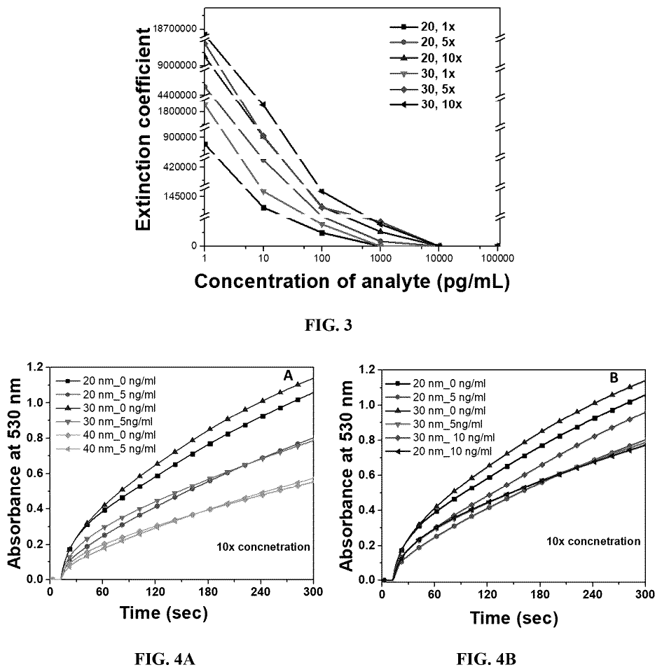

From FIG. 4A, it could be noted that 40 nm particles at 10× concentration was not able to distinguish between 0 ng/ml and about 5 ng/ml. While 30 nm AuNP labels at 10× concentration able to differentiate between 0 ng/ml and 5 ng/ml, but not 5 and 10 ng/ml (FIG. 4B). Hence 20 nm AuNP labels were taken for further experimental analysis.

The non-specific absorption (NSA) was studied by immobilizing human IgG on probe surface instead of BSA-THC.

BSA-THC immobilization on U-bent fiber optic probe was changed from covalent binding (or chemisorption) to physical adsorption or Physisorption (meaning no surface functionalization) mainly because of suspicion that there is not enough BSA-THC on the surface and hence the response was poor in FIG. 2. BSA-THC physisorbed probes gave rise to a high response as shown in FIG. 4.

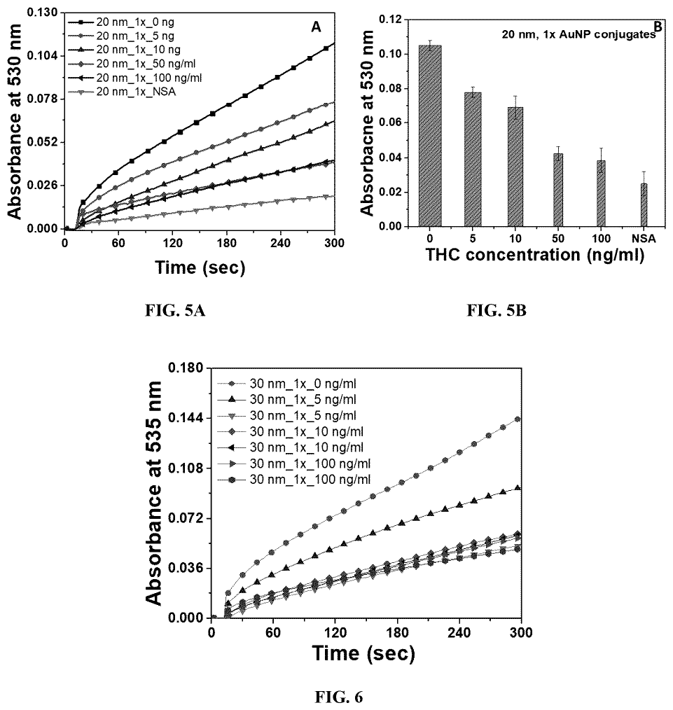

However, the subsequent study (FIG. 5) was carried out with AuNP-IgG, 20 nm, 1× concentration in order to reduce the reagent consumption and bring down the cost. In this study, BSA-THC was immobilized on to the fiber sensor surface, while anti-THC antibody was conjugated to AuNPs to form plasmonic labels. Firstly, the THC sample (30 μL, in PBS buffer) was added to AuNP labels (20 nm, 30 μL, 1× concentration) and mixed thoroughly by pipetting the mixture for 5 times and incubating the mixture for 5 min to form a plasmonic immunocomplex (AuNP-IgG-THC). Then, the sensor probe physisorbed with BSA-THC was dipped in this mixture to allow the binding of AuNP-IgG plasmonic labels with vacant THC binding sites to the BSA-THC on the probe surface. The optical intensity changes were monitored in real-time, due to absorption of the light passing through the probe caused by the plasmonic labels bound to the probe surface. This study was carried out for various THC concentrations from 0 to 100 ng/mL. The sensor response for NSA was obtained from HIgG immobilized probe instead of BSA-THC. AuNP 20 nm labels (1× conc.) were able to distinguish the THC concentration between 0 and 100 ng/mL consistently. The sensor response due to nonspecific absorption of AuNP-IgG labels is minimal, which is lower than that of 100 ng/mL THC in a sample.

Further, the THC assay was carried out with BSA-THC physisorbed probes with 30 nm AuNP plasmonic labels. The results showed that the sensor response to the THC concentrations beyond 5 ng/mL were not distinguishable, establishing that 20 nm AuNP are the optimum for THC detection in the range of 0 to 100 ng/mL. (FIG. 6).

### Example 2: Label Free Immunoassay

### Methods

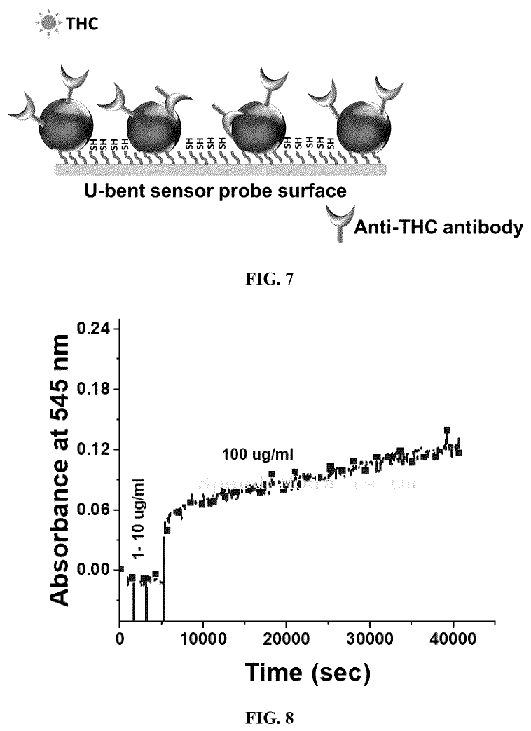

The fiber probes were functionalized using mercaptosilane and AuNPs of 40 nm were immobilized on to the silanes fiber probe (FIG. 7). The binding of AuNPs were fast and within an average time of 30 mins an absorbance response of about 2 abs. units was observed. The anti-THC antibodies were immobilized on the gold nanoparticles by exploiting affinity of amine and thiol groups present in the antibodies towards the AuNPs. The anti-THC antibody (about 50 ug/ml) immobilized overnight at about 4° C. and all the fabricated probes showed less or no response to THC concentration about 0 to 1 ug/ml. Response with about 100 ug/ml was found to be repeatable (FIG. 8).

### Example 3: Saliva Spiked with THC

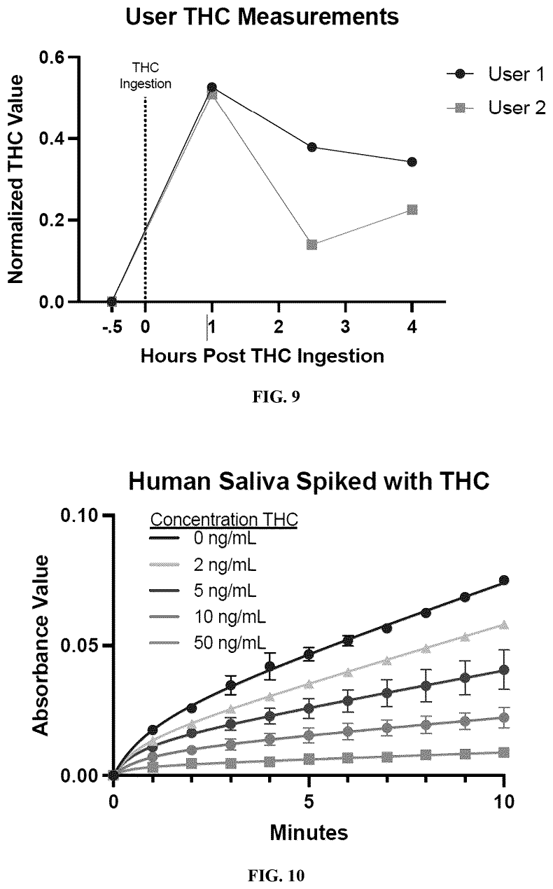

As a proof-of-the-concept for utilization of the proposed sensor towards the real-world applications, the sensor response for THC levels from the saliva of two volunteers over a duration of 9 hours were obtained. Initially, the sensor response 5 hours prior to the THC ingestion was obtained and used for normalization. Subsequently, the sensor response was obtained after 1, 2.5 and 4 hours of THC ingestion. The sensor was able to detect elevated levels of THC over the 4 hours of ingestion as shown in FIG. 9. The reduction of the THC levels over time was also picked up by the sensor. These results demonstrate the usefulness of the sensor for the identification of the subjects under the influence of THC.

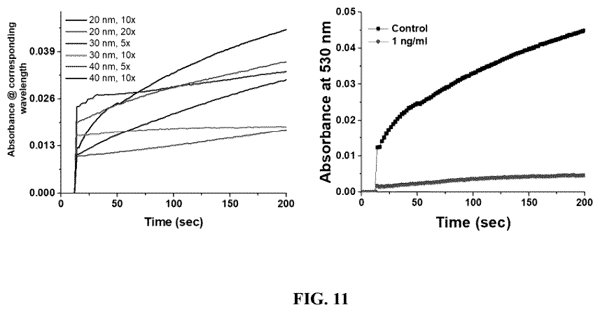

FIG. 10 shows absorbance changes detected at the probes using the labeled detection method of the present disclosure. Human saliva samples were spiked (added) with different amounts of THC and the absorbance change (decrease in absorbance) was induced in the presence of THC.

The above-disclosed features and functions, as well as alternatives, may be combined into many other different systems or applications. Various presently unforeseen or unanticipated alternatives, modifications, variations or improvements may be made by those skilled in the art, each of which is also intended to be encompassed by the disclosed embodiments.

What is claimed:

1. A method of detecting presence or absence of a cannabinoid or metabolite thereof in a biological sample, comprising:
contacting the biological sample with nanoparticle conjugated bioreceptor(s) capable of binding the cannabinoid or metabolite thereof, wherein
the nanoparticle conjugated bioreceptor(s) are either immobilized on a fiber optic sensor, or
the nanoparticle conjugated bioreceptor(s) are in solution phase; and
detecting a change in optical property of the fiber optic sensor in response to
direct binding of the cannabinoid or metabolite thereof to the nanoparticle conjugated bioreceptor(s) immobilized on the fiber optic sensor surface, or
binding of free nanoparticle conjugated bioreceptor(s) in solution phase to cannabinoid or metabolite thereof immobilized on a fiber optic sensor surface.

2. The method of claim 1, wherein the biological sample is selected from a group comprising blood, plasma, serum, saliva, urine, sweat and tears from a subject.

3. The method of claim 1, wherein the biological sample is saliva; and wherein the biological sample is optionally diluted.

4. The method of claim 3, wherein the dilution solution comprises about 10 to 100 mM Tris, about 10 to 200 mM boric acid, and about 1 to 20 mM EDTA in water and optionally about 10 to 300 mM NaCl and/or about 0.1 to 0.5 wt % lipid polymer stabilizer in water.

5. The method of claim 1, wherein the cannabinoid or metabolite thereof is selected from a group comprising tetrahydrocannabinol (THC), cannabidiol (CBD) and cannabinol (CBN) or any combination thereof.

6. The method of claim 1, wherein the bioreceptor is selected from a group comprising a peptide, a protein, antibody, an aptamer, a polymer, a hapten, a small molecule, a nucleic acid and an oligosaccharide or any combination thereof.

7. The method of claim 1, wherein the cannabinoid or metabolite thereof immobilized on the fiber optic sensor surface is immobilized in the form of a cannabinoid/metabolite-carrier conjugate.

8. The method of claim 7, wherein the carrier is selected from a group comprising Bovine Serum Albumin (BSA), serum globulins, albumins and ovalbumin or any combination thereof.

9. The method of claim 1, comprising:
contacting the biological sample with nanoparticle conjugated bioreceptor(s) capable of binding the cannabinoid or metabolite thereof, wherein the nanoparticle conjugated bioreceptor(s) are in solution phase; and
detecting a change in optical property of the fiber optic sensor in response to binding of free nanoparticle conjugated bioreceptor(s) in solution phase to cannabinoid or metabolite thereof immobilized on a fiber optic sensor surface.

10. The method of claim 1, wherein the change in optical property of the fiber optic sensor is measured using a device comprising:
a light source located proximal to one end of the optical fiber; and
a detector located proximal to another end of the optical fiber, wherein the detector is configured to sense the change in the optical property of light that traverses through the optical fiber when the probe region is exposed to the biological sample comprising the cannabinoid or metabolite thereof.

11. A test kit for detecting a cannabinoid or metabolite thereof in a biological sample, comprising:
the device is as described in claim 10;
means for sample collection and optionally, quantification and/or storage;
and one or more of:
a vial or container comprising a blocking agent;
a second vial or container comprising a sample diluting solution or components thereof; and
an instruction manual.

12. The test kit of claim 11, wherein the blocking agent is selected from a group comprising bovine serum albumin (BSA), casein, sericin, lipids, detergent, poly(ethylene glycol) and polyvinylpyrrolidone (PVP) or any combination thereof.

13. The test kit of claim 11, wherein the sample diluting solution comprises about 10 to 100 mM Tris, about 10 to 200 mM boric acid, and about 1 to 20 mM EDTA in water and optionally about 10 to 300 mM NaCl and/or about 0.1 to 0.5 wt % lipid polymer stabilizer in water.

14. Use of the device as defined in claim 10 for the qualitative and/or quantitative detection of cannabinoid or metabolite thereof in a biological sample.

15. Use of the test kit of claim 11 for the qualitative and/or quantitative detection of cannabinoid or metabolite thereof in a biological sample.
